(19) United States
(12) Patent Application Publication
(10) Pub. No.: US 2013/0345296 A1
(43) Pub. Date: Dec. 26, 2013

(54) Compositions Comprising a Taxane for Coating Medical Devices

(71) Applicants: Torsten Heilmann, Bad Klosterlausnitz (DE); Heiko Noack, Halle/Saale (DE); Christian Richter, Oberw-rschnitz (DE); Sabine Post, Berlin (DE)

(72) Inventors: Torsten Heilmann, Bad Klosterlausnitz (DE); Heiko Noack, Halle/Saale (DE); Christian Richter, Oberw-rschnitz (DE); Sabine Post, Berlin (DE)

(73) Assignee: AVIDAL VASCULAR GMBH, Halle (Saale) (DE)

(21) Appl. No.: 13/819,538

(22) PCT Filed: Aug. 31, 2011
(86) PCT No.: PCT/EP2011/004385

§ 371 Date: Sep. 13, 2013

Related U.S. Application Data
(60) Provisional application No. 61/378,594, filed on Aug. 31, 2010.

(51) Int. Cl. A61K 31/337; A61K 31/27; A61K 31/198

(52) U.S. Cl. CPC A61K 31/337; A61K 31/198; A61K 31/27
USPC 514/449

(57) ABSTRACT

The present invention relates a pharmaceutical composition comprising a taxane and an inhibitory compound capable of inhibiting the enzyme acetaldehyde dehydrogenase e.g. disulfiram. The pharmaceutical compositions of the invention can be used to coat medical devices such as implants. Such implant can be an expansible hollow part, having at least one opening, which consists of an elastic biocompatible material that comprises elongated micro-cavities in its surface. Medical devices coated with a pharmaceutical composition of the invention exhibit an enhanced transfer of taxane from the surface of said medical device into the target tissue.

The present invention relates a pharmaceutical composition comprising a taxane and an inhibitory compound capable of inhibiting the enzyme acetaldehyde dehydrogenase. The pharmaceutical compositions of the invention can be used to coat medical devices such as implants. Such implant can be an expansible hollow part, having at least one opening, which consists of an elastic biocompatible material that comprises elongated micro-cavities in its surface. Medical devices coated with a pharmaceutical composition of the invention exhibit an enhanced transfer of taxane from the surface of said medical device into the target tissue.

### BACKGROUND OF THE INVENTION

Taxanes are poorly water soluble alkaloids. Taxol has originally been isolated from several species of Western Yew. Taxanes exhibit antimitotic properties and are effective therapeutic agents. For example, taxol has been shown to be active against leukemia, colon, breast, melanoma, sarcomas, and Lewis lung tumor systems (see e.g. Tarr et al. (1987) Pharm. Res. 4:162-165; Horwitz (1992) TIPS 13:134-136). In vitro studies indicate that concentrations of taxol of 0.1-10.0 μg/ml stabilize microtubules, thus disrupting normal cell division (Rowinsky et al. (1990) J. Natl. Cancer Inst. 82:1247-1259).

Taxanes have also clinically been used to coat medical devices. For example for the treatment of a stenosis, implantation of taxane-coated vessel grafts such as stents have become a well-established surgical intervention. In this context, the taxane can reduce so-called restenosis (recurrent stenosis), i.e. the reocclusion of the vessel is a frequently occurring complication. There's no exact definition of the term restenosis to be found in literature. The most frequently used morphological definition of restenosis defines restenosis as a reduction of the vessel diameter to less than 50% of the normal value subsequent to successful PTA (percutaneous transluminal angioplasty). In practice, clinical deterioration in a patient is often considered a sign for the occurrence of restenosis in the previously treated vessel section.

Restenosis following stent implantation is one of the major causes for further hospitalization. Vessel traumas induced during stent implantation cause inflammatory reactions which play a decisive role in the healing process during the first seven days. As mentioned, it has also been found that stents provided with a taxane-eluting coating counteract restenosis.

But also so-called “biological stenting” may be performed using only a coated catheter balloon without any stent, i.e. the vessels are dilated at a constricted site by the dilatation of a coated catheter balloon, wherein, while the catheter balloon is dilated for a short period of time, a sufficient amount of pharmacological agent is transferred to the vessel wall to avoid re-constriction or reocclusion of the vessel due to the dilatation of the vessel and the delivery of active agents. Such coated catheter balloons can be manufactured by directly applying the taxane to the surface of the balloon e.g. using known methods for spray coating as described in WO 2004/006976 A1.

One complication that frequently arises when using taxane coated medical devices is that the taxane is transferred into tissue only slowly and to a partial extent, due to its poor solubility in water and many oils. In an attempt to increase taxol's solubility and develop more feasible clinical formulations, investigators have modified the structure of taxanes, e.g. acylated carbons at the 7-position and 10-position of the taxene ring. These efforts have yielded compounds that retain their biological activity but are expensive and have further deficits such as their stability.

Thus, there is a need for improved therapeutic compositions comprising taxane that can be used to e.g. coat medical devices and that can be manufactured with relatively small effort and costs and which provide an improved transfer of the active substance, i.e. taxane to the target tissue compared with taxane-containing compositions available in the prior art.

### SUMMARY OF THE INVENTION

To solve above-mentioned problems and further problems associated with the prior art compositions, the present invention provides A pharmaceutical composition comprising a taxane and an inhibitory compound capable of inhibiting the enzyme acetaldehyde dehydrogenase.

Further provided is a medical device comprising the pharmaceutical composition according to the invention.

In a further aspect the invention provides a pharmaceutical according to the invention for use in the treatment or prevention of diseases associated with or caused by hyperproliferation of cells.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In the following definitions of the chemical terms: “alkyl”, “heteroalkyl”, “cycloalkyl”, “heterocycloalkyl”, “alicyclic system”, “aryl”, “aralkyl”, “heteroaryl”, “heteroaralkyl”, “alkenyl”, “cycloalkenyl”, “alkynyl” and “optionally substituted” are provided. These terms will in each instance of its use in the remainder of the specification have the respectively defined meaning and preferred meanings.

The term “alkyl” refers to a saturated straight or branched carbon chain. Preferably, the chain comprises from 1 to 10 carbon atoms, i.e. 1, 2, 3, 4, 5, 6, 7, 8, 9 or 10 e.g. methyl, ethyl, propyl, iso-propyl, butyl, iso-butyl, tert-butyl, pentyl or hexyl, heptyl, or octyl. Alkyl groups are optionally substituted.

The term “heteroalkyl” refers to a saturated straight or branched carbon chain. Preferably, the chain comprises from 1 to 9 carbon atoms, i.e. 1, 2, 3, 4, 5, 6, 7, 8, 9 e.g. methyl, ethyl, propyl, iso-propyl, butyl, iso-butyl, sec-butyl, tert-butyl, pentyl or hexyl, heptyl, octyl, which is interrupted one or more times, e.g. 1, 2, 3, 4, 5, with the same or different heteroatoms. Preferably the heteroatoms are selected from O, S, and N, e.g. —O—CH₃, —S—CH₃, —CH₂—O—CH₃, —CH₂—O—C₂H₅, —CH₂—S—CH₃, —CH₂—S—C₂H₅, —C₂H₄—O—CH₃, —C₂H₄—O—C₂H₅, —C₂H₄—S—CH₃, —C₂H₄—S—C₂H₅ etc. Heteroalkyl groups are optionally substituted.

The terms “cycloalkyl” and “heterocycloalkyl”, by themselves or in combination with other terms, represent, unless otherwise stated, cyclic versions of “alkyl” and “heteroalkyl”, respectively, with preferably 3, 4, 5, 6, 7, 8, 9 or 10 atoms forming a ring, e.g. cyclopropyl, cyclobutyl, cyclopentyl, cyclohexyl, cycloheptyl, cyclooctyl etc. The terms “cycloalkyl” and “heterocycloalkyl” are also meant to include bicyclic, tricyclic and polycyclic versions thereof. If more than one cyclic ring is present such as in bicyclic, tricyclic and polycyclic versions, then these rings may also comprise one or more aryl- or heteroaryl ring. The term “heterocycloalkyl” preferably refers to a saturated ring having five members of which at least one member is a N, O or S atom and which optionally contains one additional O or one additional N; a saturated ring having six members of which at least one member is a N, O or S atom and which optionally contains one additional O or one additional N or two additional N atoms; or a saturated bicyclic ring having nine or ten members of which at least one member is a N, O or S atom and which optionally contains one, two or three additional N atoms. “Cycloalkyl” and “heterocycloalkyl” groups are optionally substituted. Additionally, for heterocycloalkyl, a heteroatom can occupy the position at which the heterocycle is attached to the remainder of the molecule. Preferred examples of cycloalkyl include cyclopropyl, cyclobutyl, cyclopentyl, cyclohexyl, cycloheptyl, cyclooctyl, cyclononyl, cyclodecyl, spiro[3,3]heptyl, spiro[3,4]octyl, spiro[4,3]octyl, spiro[3,5]nonyl, spiro[5,3]nonyl, spiro[3,6]decyl, spiro[6,3]decyl, spiro[4,5]decyl, spiro[5,4]decyl, bicyclo[4.1.0]heptyl, bicyclo[3.2.0]heptyl, bicyclo[2.2.1]heptyl, bicyclo[2.2.2]octyl, bicyclo[5.1.0]octyl, bicyclo[4.2.0]octyl, octahydro-pentalenyl, octahydro-indenyl, decahydro-azulenyl, adamantly, or decahydro-naphthalenyl. Examples of heterocycloalkyl include 1-(1,2,5,6-tetrahydropyridyl), 1-piperidinyl, 2-piperidinyl, 3-piperidinyl, 4-morpholinyl, 3-morpholinyl, 1,8 diaza-spiro-[4,5] decyl, 1,7 diaza-spiro-[4,5] decyl, 1,6 diaza-spiro-[4,5] decyl, 2,8 diaza-spiro[4,5] decyl, 2,7 diaza-spiro[4,5] decyl, 2,6 diaza-spiro[4,5] decyl, 1,8 diaza-spiro-[5,4] decyl, 1,7 diaza-spiro-[5,4] decyl, 2,8 diaza-spiro-[5,4] decyl, 2,7 diaza-spiro[5,4] decyl, 3,8 diaza-spiro[5,4] decyl, 3,7 diaza-spiro[5,4] decyl, 1-aza-7,11-dioxo-spiro[5,5] undecyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The term “alicyclic system” refers to mono, bicyclic, tricyclic or polycyclic version of a cycloalkyl or heterocycloalkyl comprising at least one double and/or triple bond. However, an alicyclic system is not aromatic or heteroaromatic, i.e. does not have a system of conjugated double bonds/free electron pairs. Thus, the number of double and/or triple bonds maximally allowed in an alicyclic system is determined by the number of ring atoms, e.g. in a ring system with up to 5 ring atoms an alicyclic system comprises up to one double bond, in a ring system with 6 ring atoms the alicyclic system comprises up to two double bonds. Thus, the “cycloalkenyl” as defined below is a preferred embodiment of an alicyclic ring system. Alicyclic systems are optionally substituted.

The term “aryl” preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphtyl or anthracenyl. The aryl group is optionally substituted.

The term “aralkyl” refers to an alkyl moiety, which is substituted by aryl, wherein alkyl and aryl have the meaning as outlined above. An example is the benzyl radical. Preferably, in this context the alkyl chain comprises from 1 to 8 carbon atoms, i.e. 1, 2, 3, 4, 5, 6, 7, or 8, e.g. methyl, ethyl methyl, ethyl, propyl, iso-propyl, butyl, iso-butyl, sec-butenyl, tert-butyl, pentyl or hexyl, pentyl, octyl. The aralkyl group is optionally substituted at the alkyl and/or aryl part of the group.

The term “heteroaryl” preferably refers to a five or six-membered aromatic monocyclic ring wherein at least one of the carbon atoms are replaced by 1, 2, 3, or 4 (for the five membered ring) or 1, 2, 3, 4, or 5 (for the six membered ring) of the same or different heteroatoms, preferably selected from O, N and S; an aromatic bicyclic ring system wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system wherein 1, 2, 3, 4, 5, or 6 carbon atoms of the 13, 14, 15, or 16 carbon atoms have been replaced with the same or different heteroatoms, preferably selected from O, N and S. Examples are oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indolyl, isoindolyl, benzothiophenyl, 2-benzothiophenyl, 1H-indazolyl, benzimidazolyl, benzoxazolyl, indoxazinyl, 2,1-benzisoxazoyl, benzothiazolyl, 1,2-benzisothiazolyl, 2,1-benzisothiazolyl, benzotriazolyl, quinolinyl, isoquinolinyl, quinoxalinyl, quinazolinyl, 1,2,3-benzotriazinyl, or 1,2,4-benzotriazinyl.

The term “heteroaralkyl” refers to an alkyl moiety, which is substituted by heteroaryl, wherein alkyl and heteroaryl have the meaning as outlined above. An example is the 2-alklypyridinyl, 3-alkylpyridinyl, or 2-methylpyridinyl. Preferably, in this context the alkyl chain comprises from 1 to 8 carbon atoms, i.e. 1, 2, 3, 4, 5, 6, 7, or 8, e.g. methyl, ethyl methyl, ethyl, propyl, iso-propyl, butyl, iso-butyl, sec-butenyl, tert-butyl, pentyl or hexyl, pentyl, octyl. The heteroaralkyl group is optionally substituted at the alkyl and/or heteroaryl part of the group.

The terms “alkenyl” and “cycloalkenyl” refer to olefinic unsaturated carbon atoms containing chains or rings with one or more double bonds. Examples are propenyl and cyclohexenyl. Preferably, the alkenyl chain comprises from 2 to 8 carbon atoms, i.e. 2, 3, 4, 5, 6, 7, or 8, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl, octenyl. The term also comprises CH₂, i.e. methenyl, if the substituent is directly bonded via the double bond. Preferably the cycloalkenyl ring comprises from 3 to 14 carbon atoms, i.e. 3, 4, 5, 6, 7, 8, 9, 10, 11, 12, 13 or 14, e.g. cyclopropenyl, cyclobutenyl, cyclopentenyl, cyclohexenyl, cycloheptenyl, cyclooctyl, cyclononenyl, cyclodecenyl, spiro[3,3]heptenyl, spiro[3,4]octenyl, spiro[4,3]octenyl, spiro[3,5]nonenyl, spiro[5,3]nonenyl, spiro[3,6]decenyl, spiro[6,3]decenyl, spiro[4,5]decenyl, spiro[5,4]decenyl, bicyclo[4.1.0]heptenyl, bicyclo[3.2.0]heptenyl, bicyclo[2.2.1]heptenyl, bicyclo[2.2.2]octenyl, bicyclo[5.1.0]octenyl, bicyclo[4.2.0]octenyl, hexahydro-pentalenyl, hexahydro-indenyl, octahydro-azulenyl, or octahydro-naphthalenyl.

The term “alkynyl” refers to unsaturated carbon atoms containing chains or rings with one or more triple bonds. An example is the propargyl radical. Preferably, the alkynyl chain comprises from 2 to 8 carbon atoms, i.e. 2, 3, 4, 5, 6, 7, or 8, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl, octynyl.

The term “optionally substituted” in each instance if not further specified refers to between 1 and 10 substituents, e.g. 1, 2, 3, 4, 5, 6, 7, 8, 9, or 10 substituents which are in each instance preferably independently selected from the group consisting of halogen, in particular F, Cl, Br or I; —NO₂, —CN, —OR′, —NR′R″, —(CO)OR′, —(CO)OR′″, —(CO)NR′R″, —NR′COR″″, —NR′COR′, —NR″CONR′R″, —NR″SO₂A, —COR′″; —SO₂NR′R″, —OOCR′″, —CR′″R″″OH, —R′″OH, ═O, and —E;

R′ and R″ is each independently selected from the group consisting of hydrogen, alkyl, alkenyl, alkynyl, -OE, cycloalkyl, heterocycloalkyl, aryl, heteroaryl, and aralkyl or together form a heteroaryl, or heterocycloalkyl; optionally substituted;

R′″ and R″″ is each independently selected from the group consisting of alkyl, alkenyl, alkynyl, cycloalkyl, heterocycloalkyl, alkoxy, aryl, aralkyl, heteroaryl, and —NR′R″;

E is selected from the group consisting of alkyl, alkenyl, cycloalkyl, alkoxy, alkoxyalkyl, heterocycloalkyl, an alicyclic system, aryl and heteroaryl; optionally substituted;

If two or more radicals can be selected independently from each other, then the term “independently” means that the radicals may be the same or may be different.

One object of the present invention is to provide compositions which permit an improved transfer of a taxane from a medical device coated with the composition to a target tissue. Such compositions can for example be applied to vessel grafts such as stents but also to inflatable and expansible medical devices such as a balloon catheter or an expansible hollow part that can be used on the balloon of a balloon catheter. It is one unexpected finding of the present invention, that if an expansible hollow part, e.g. an expansible tube made of polyisoprene is coated with the pharmaceutical compositions of the invention, that up to 14-fold more taxane is transferred from the coat to the target tissue as compared to when comparable prior art medical devices comprising paclitaxel are used.

Thus, such coated devices will provide an effective means to counteract e.g. neointimal hyperplasia, restenosis, inflammation and thrombosis, solving the aforementioned problems with conventionally coated medical devices while providing the possibility of a controlled and improved delivery of the therapeutic substance.

Thus, in a first aspect, the invention provides A pharmaceutical composition comprising a taxane and an inhibitory compound capable of inhibiting the enzyme acetaldehyde dehydrogenase. As shown in the examples below, a medical device coated with a composition according to the first aspect of the invention was capable of transferring up to about 14-fold more paclitaxel into target tissue than prior art balloon catheters that were formulated with a matrix of pure paclitaxel (e.g. compare FIG. 1 “WOMBAT IIa” with “ELU”). Taxanes are diterpenes which are mitotic inhibitors and include e.g. paclitaxel (Taxol) and docetaxel.

Acetaldehyde dehydrogenases (EC 1.2.1.10) are dehydrogenase enzymes which catalyze the conversion of acetaldehyde into acetic acid. The oxidation of acetaldehyde to acetate can be summarized as follows:

CH₃CHO+NAD⁺+CoA→acetyl-CoA+NADH+H⁺

In humans, there are three known genes which encode this enzymatic activity, ALDH1A1, ALDH2, and the more recently discovered ALDH1B1 (also known as ALDH5).

These enzymes are members of the larger class of aldehyde dehydrogenases. Compounds that inhibit acetaldehyde dehydrogenases are well known in the art and are further described below. For example, acetaldehyde dehydrogenase is inhibited by fatty acyl-CoA derivatives. Also the average skilled person can determine without undue burden, whether a compound inhibits an acetaldehyde dehydrogenase enzyme. For example, an assay for acetaldehyde dehydrogenase activity can be based on the conversion of radioactive acetaldehyde to acetate as has been described in Liu et al., 1965, Plant Physiol. 40: 1261-1268. Alternatively or additionally, the aldehyde concentration can also be monitored spectroscopically.

In a preferred embodiment of the first aspect, the inhibitory compound has the structure according to formula (I):

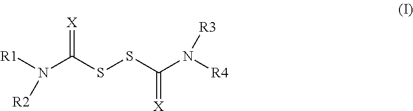

wherein

X is O or S; preferably S; and

R¹-R⁴ are each individually selected from the group consisting of C₁-C₆-alkyl (e.g. methyl, ethyl, propyl, iso-propyl, butyl, iso-butyl, tert-butyl, pentyl or hexyl), C₂-C₆-alkenyl (preferably C₁, C₂, C₃, C₄, C₅ or C₆-alkenyl), C₂-C₈-alkynyl (preferably C₁, C₂, C₃, C₄, C₅, C₆, C₇ or C₈-alkynyl), —(CH₂)ₙC(O)OH, —(CH₂)ₙC(O)OR⁵, —(CH₂)ₙOH, —(CH₂)ₙOR⁵, —(CH₂)ₙ-cycloalkyl, —(CH₂)ₙC(O)NH₂, —(CH₂)ₙC(O)NHR⁵, —(CH₂)ₙ-aryl, —(CH₂)ₙ-heteroaryl and —(CH₂)ₙNHR⁵; optionally substituted;

R⁵ is selected from the group consisting of hydrogen, C₁-C₆-alkyl (e.g. methyl, ethyl, propyl, iso-propyl, butyl, iso-butyl, tert-butyl, pentyl or hexyl), C₂-C₆-alkenyl (preferably C₁, C₂, C₃, C₄, C₅ or C₆-alkenyl), C₂-C₆-alkynyl (preferably C₁, C₂, C₃, C₄, C₅ or C₆-alkynyl), —(CH₂)ₙ-cycloalkyl and —(CH₂)ₙ-aryl; optionally substituted;

and

n is in each instance selected from 0, 1 and 2.

In a preferred embodiment of the pharmaceutical composition of the invention, the inhibitory compound is coprin or disulfiram. Disulfiram (1,1′,1″,1′″-[disulfanediylbis(carbonothioylnitrilo)]tetraethane, tetraethylthiuramdisulfid) is a drug used to e.g. support the treatment of chronic alcoholism by producing an acute sensitivity (hangover) to alcohol. Disulfiram blocks the enzyme acetaldehyde dehydrogenase.

In a further preferred embodiment of the pharmaceutical composition according to the invention, the molar ratio between the taxane and said inhibitory compound is between 3.5:1 and 0.07:1, preferably between 2:1 and 0.2:1 and most preferably between 1.5:1 and 0.3:1. Further preferred ratios are also shown in the examples and FIG. 1.

In a preferred embodiment of the pharmaceutical composition according to the invention said taxane is paclitaxel or docetaxel.

It was shown that the addition of a polyol to the composition of the invention not only improved the quality of the coating when sprayed onto a medical device but it also increased the amount of taxane that was transferred to the target tissue. Thus in a further preferred embodiment, the pharmaceutical composition of the invention further comprises a polyol.

As used herein, a polyol refers to an alcohol preferably an alkyl alcohol, aryl alcohol, a heterocyclic alcohol or a cycloalkyl alcohol, containing multiple, preferably between 2 and 20 (i.e. 2, 3, 4, 5, 6, 7, 8, 9, 10, 11, 12, 13, 14, 15, 16, 17, 18, 19 or 20), more preferably between 2 and 5 (i.e. 2, 3, 4 or 5) and most preferably between 2 and 3 hydroxyl groups. In one embodiment of the first and second aspect of the invention the polyol is an alcohol with between 2 and 10 (i.e. 2, 3, 4, 5, 6, 7, 8, 9 or 10) hydroxyl groups.

Thus, suitable polyols that can be used in the composition of the invention include e.g. pentaerythritol, erythritol, glycol (preferably ethylene glycol) and glycerol. In a most preferred embodiment glycerol is used in the compositions of the invention.

In a further preferred embodiment of the compositions of the invention the molar ratio between the taxane and said polyol (preferably glycerol) is between 1:3 to 1:250 and preferably between 1:6 to 1:120 and most preferably between 1:25 to 1:35. These ratios proved to be most effective to provide a stable and even coating on medical devices and at the same time enhanced the transfer of the taxane from the medical device into the target tissue. With ratios smaller than 1:3 only a poor transfer of paclitaxel into tissue was observed. With ratios exceeding 1:250 paclitaxel was washed off of the medical device in the blood vessel prematurely. When preparing a coating solution, wherein the ingredients of the compositions of the invention are preferably dissolved in ethanol (see also examples below), it is preferred that such coating solution comprises glycerol in the final amount of between 3% and 7%, more preferably between 4% and 6% and most preferably in the amount of about 5% (v/v). Such coating solutions are also within the ambit of the invention.

The pharmaceutical composition according to the invention may in preferred embodiments also comprise an additional active compound selected from the following group: abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxycyclo phosphamide idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mitoxantrone, mycophenolate mofetil, [beta]-1apachone, podophyllotoxin, podophyllic acid 2-ethylhydrazide, molgramostim (rhuGM-CSF), peginterferon [alpha]-2b, lenograstim (r-HuG-CSF), macrogol, selectin (cytokine antagonist), cytokinin inhibitors, COX-2 inhibitor, angiopeptin, monoclonal antibodies inhibiting muscle cell proliferation, bFGF antagonists, probucol, prostaglandins, 1-hydroxy-11-methoxycanthin-6-one, scopoletin, NO donors, pentaerythrityl tetranitrate and sydnoimines, S-nitroso derivatives, tamoxifen, staurosporine, [beta]-estradiol, [alpha]-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof, 6-[alpha]-hydroxy-paclitaxel, taxoteres, mofebutazone, lonazolac, lidocaine, ketoprofen, mefenamic acid, piroxicam, meloxicam, penicillamine, hydroxychloroquine, sodium aurothiomalate, oxaceprol, [beta]-sitosterol, myrtecaine, polidocanol, nonivamide, levomenthol, ellipticine, D-24851 (Calbiochem), colcemid, cytochalasin A-E, indanocine, nocodazole, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF-1, active agents from the group of antibiotics, cefadroxil, cefazolin, cefaclor, cefoxitin, tobramycin, gentamicin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon [alpha], [beta] and [gamma], histamine antagonists, serotonin blockers, apoptosis inhibitors, apoptosis regulators, halofuginone, nifedipine, tocopherol, tranilast, molsidomine, tea polyphenols, epicatechin gallate, epigallocatechin gallate, leflunomide, etanercept, sulfasalazine, dicloxacillin, tetracycline, triamcinolone, mutamycin, procainimide, retinoic acid, quinidine, disopyrimide, flecamide, propafenone, sotalol, natural and synthetically obtained steroids such as bryophyllin A, inotodiol, maquiroside A, ghalakinoside, mansonine, strebloside, hydrocortisone, betamethasone, dexamethasone, non-steroidal substances (NSAIDS) such as fenoprofen, fenoprofen, ibuprofen, indomethacin, naproxen, phenylbutazone, antiviral agents, acyclovir, ganciclovir zidovudine, clotrimazole, flucytosine, griseofulvin, ketoconazole, miconazole, nystatin, terbinafine, antiprotozoal agents, chloroquine, mefloquine, quinine, natural terpenoids, hippocaesculin, barringtogenol-C21-angel ate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A,B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6[alpha]-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-[beta]-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B and zeorin.

In a further aspect the invention provides a medical device comprising a pharmaceutical composition according to the invention as described above. Particularly preferred medical devices are selected from the group consisting of implants such as stents, a balloon catheter (in particular for percutaneous transluminal coronary angioplasty) and an expansible hollow part. The expansible hollow part is useful to be e.g. used on a balloon catheter. If the medical device is an expansible hollow part, it preferably has at least one and more preferably at leas two openings (e.g. a tube) and it consists of an elastic biocompatible material that comprises micro-cavities in its surface that are preferably elongated. In a further preferred embodiment of the medical device, the medical device is a hollow part as described, wherein more than 50% of a pharmaceutical composition of the invention is located in said micro-cavities. Preferably, the material of the expansible hollow part consists, comprises or essentially consists of a material selected from the group consisting of:
natural rubber, polyisoprene, a copolymer of isobutylene and isoprene, a halogenated butyl rubber, a polybutadiene, a styrene-butadiene rubber, a copolymer of polybutadiene and acrylonitrile, a hydrogenated nitrile rubber, a chloroprene rubber, a polychloroprene, latex, a neoprene, a baypren, latex, parylene, polyvalerolactone, poly-ε-decalactone, polylactic acid, polyglycol acid, polylactide, polyglycolide, co-polymer of polylactide and polyglycolide, poly-ε-caprolactone, polyhydroxy butyric acid, polyhydroxybutyrate, polyhydroxyvalerate, polyhydroxybutyrate-co-valerate, poly(1,4-dioxan-2,3-dione), poly(1,3-dioxan-2-one), poly-para-dioxanone, polyanhydride, polymaleicacidanhydride, polyhydroxymethacrylate, fibrin, polycyanoacrylate, polycaprolactondimethylacrylate, poly-β-maleic acid, polycaprolactonbutylacrylate, multiblockpolymers made of oligocaprolactondiole and oligodioxanondiole, polyetherestermultiblockpolymers made from PEG and polybutylenterephtalate, polypivotolactone, poly-glycolic acid trimethylcarbonate polycaprolactonglycolide, poly(g-ethylglutamate), poly(dth-iminocarbonate), poly(dte-co-dt-carbonat), poly(bisphenol A-iminocarbonate), polyorthoester, poly-glycolic acid-trimethylcarbonate, polytrimethylcarbonate polyiminocarbonate, poly(n-vinyl)-pyrrolidone, polyvinylalcohols, polyesteramide, glycolized polyester, polyphosphoester, polyphosphazene, poly(p-carboxyphenoxy)propane], polyhydroxypentanoic acid, polyethylenoxidpropylenoxid, polyurethane, polyurethane comprising amino acids, polyetherester like polyethyleneoxide, polyalkeneoxalate, polyorthoester, lipids, carrageenane, fibrinogen, starch, collagene, protein-based polymers, polyaminoacids, zein, polyhydroxyalkanoate, pectic acid, actinic acid, carboxymethylsulfate, albumine, hyaluronic acid, chitosane, heparanesulfate, heparine, chondroitinsulfate, dextrane, β-cyclodextrine, copolymers comprising PEG and polypropyleneglycole, gummi arabicum, guar, gelatine, collagen-n-hydroxysuccinimide, phospholipids, polyacrylic acid, polyacrylate, polymethylmethacrylate, polybutylmethacrylate, polyacrylamide, polyacrylonitrile, polyamide, polyetheramide, polyethyleneamine, polyimide, polycarbonate, polycarbourethane, polyvinylketone, polyvinylhalogenide, polyvinylidenhalogenide, polyvinylether, polyisobutylene, aromatic compounds comprising a polyvinyl functional group, polyvinylester, polyvinylpyrollidone, polyoxymethylene, polytetramethyleneoxide, polyethylen, polypropylen, polytetrafluorethylen, polyetherurethane, silicon-polyetherurethane, silicon-polyurethane, silicon-polycarbonat-urethane, polyolefin-elastomers, epdm-rubber, fluorosilicone, carboxymethylchitosane, polyaryletheretherketone, polyetheretherketone, polyethylenterephtalate, polyvalerate, carboxymethylcellulose, cellulose, rayon, rayontriacetate, cellulosenitrate, celluloseacetate, hydroxyethylcellulose, cellulosebutyrate, celluloseacetatebutyrate, ethylvinylacetate, polysulfone, epoxy-resin, abs-resin, silicone like polysiloxane, polydimethylsiloxane, polyvinylhalogens, cellulose-ether, cellulose-triacetate, copolymers mixtures and derivatives thereof.

In a most preferred embodiment, the elastic biocompatible material of the expansible hollow part is polyisoprene or latex, especially Guayule (Parthenium argentatum) latex which is hypoallergenic.

As used herein “micro-cavity” refers to either a hole or a furrow such as a groove. The cross-section of said furrow can have any shape. If the micro-cavity is a hole, the hole is a pit that can also have any shape but a micro-cavity that is a hole is not a perforation, i.e. not an opening connecting the outer and inner surface of the hollow part of the invention. Thus, if an expansible hollow part of the invention comprises micro-cavities these cavities according to the invention do not penetrate the material of the hollow part, e.g. to connect any outer surface with an inner surface of the material. This is advantageous since the cavities do not substantially weaken the material which is thus, resilient against mechanical stress and can undergo a substantial expansion without tear. For the same reason it is preferred that the surface of the expansible hollow part may preferably not comprise a plurality of perforations through which liquid can penetrate when the expansible hollow part of the invention is in its expanded state. It is, thus also preferred that the surface of the expansible hollow part of the invention is substantially impermeable to liquid and/or gas. The micro-cavities in the surface of the expansible hollow part can be generated by e.g. blasting the surface with sand, glass beads, or water. Preferably the micro-cavities are cut utilizing laser or plasma eroding techniques.

If the micro-cavities are elongated then they are preferably selected from the group consisting of crescent-shaped furrows, sinuous furrows, circular furrows, elliptical furrows, furrows comprising one or more bends, straight furrows, bifurcated furrows and combinations thereof. In one preferred embodiment, the elongated micro-cavities have a length of not more than 0.1 mm, 0.2 mm, 0.3 mm, 0.4 mm, 0.5 mm, 0.6 mm, 0.7 mm, 0.8 mm, 0 9 mm, 1 mm, 2 mm, 3 mm, 4 mm, 5 mm, or more than 6 mm Irrespective of whether the micro-cavities are holes or furrows, it is preferred that their depth is between 5 μm and 500 μm, which allows good mechanical strength and good loading capacities of the biologically active substance which is as further detailed below preferably loaded into the micro-cavities. As already mentioned, it is preferred that the cavities do not penetrate the surface of the hollow part and, thus, the cavity depth is preferably smaller than the thickness of the wall of the expansible hollow part of the invention.

As used herein “expansible” refers to the ability of the material of the expansible hollow part according to the invention to reversibly expand its surface when exposed to mechanical stress, i.e. a force causing deformation. Thus, the surface will return to its original, i.e. “relaxed” configuration, when the stress is removed. As used herein “relaxed” means in the absence of any external forces except the average atmospheric pressure present on earth at an altitude of between zero and 500 m above sea level. If in a preferred embodiment the expansible hollow part of the invention is a tubular structure, it is preferred that “expansible” means that the tubular structure can be reversibly expanded in its circumference. In a preferred embodiment, the expansible hollow part of the invention is expansible to at least 110%, 115%, 120%, 140%, 160%, 180%, 200%, 400%, 600%, 800%, 1000%, 1200%, 1400%, 1600%, 1800% or to at least 2000% of the circumference of its non-expanded state. Preferably the expansible hollow part according to the invention has an inner diameter smaller than 3, 2.5, 2.0, 1.5, 1, 0.9, 0.8, 0.7, 0.6, 0.5, 0.4, 0.3, 0.2 or smaller than 0.1 cm in its non-expanded state. Most preferably, the inner diameter is smaller than 1 cm in its non-expanded state when measured at its narrowest section.

Preferably the wall thickness of the expansible hollow part of the invention is between 200 μm and 1000 μm and the hollow part comprises micro-cavities that have a maximal depth of 100 nm, 1 μm, 10 μm, 20 μm, 30 μm, 40 μm, 50 μm, 60 μm, 70 μm, 80 μm, 90 μm or 100 μm.

Preferred expansible hollow parts that can be used according to the invention, their making and coating has also been described in WO 2009/121565 incorporated herein by reference in its entirety.

Expansible hollow parts, preferably in tube-form, coated with a composition of the invention can be used with a balloon catheter which is covered by the expansible hollow part. Thus, when the balloon catheter is inflated, the expansible hollow part of the invention is expanded and the paclitaxel is transferred to the vessel or organ wall by contact. This allows an efficient transfer of the biologically active substance from the hollow part to the tissue.

In a further preferred embodiment of the medical device of the invention, at least part of said device is coated with said pharmaceutical composition such that the average taxane concentration on said coated surface is between 2 and 10 μg taxane/mm², preferably between 2 and 6 μg taxane/mm² and most preferably between 3 and 5 μg taxane/mm².

In a further aspect the invention relates to a pharmaceutical composition according to the invention for use in the treatment or prevention of diseases associated with or caused by hyperproliferation of cells. In this context it is preferred that the disease is selected from stenosis, restenosis, stricture, defective bypass craft, thrombosis, dissection, tumor, calcification, arteriosclerosis, inflammation, autoimmune response, necrosis, injured anastomosis, lesion, allergy, wart, hyperproliferation, infection, scald, edema, coagulation, cicatrization, burn, frostbite and lymphangitis.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following figures and examples are merely illustrative of the present invention and should not be construed to limit the scope of the invention as indicated by the appended claims in any way.

### BRIEF DESCRIPTION OF THE FIGURES

FIG. 1: Efficiency of transfer of the therapeutic substance paclitaxel into tissue

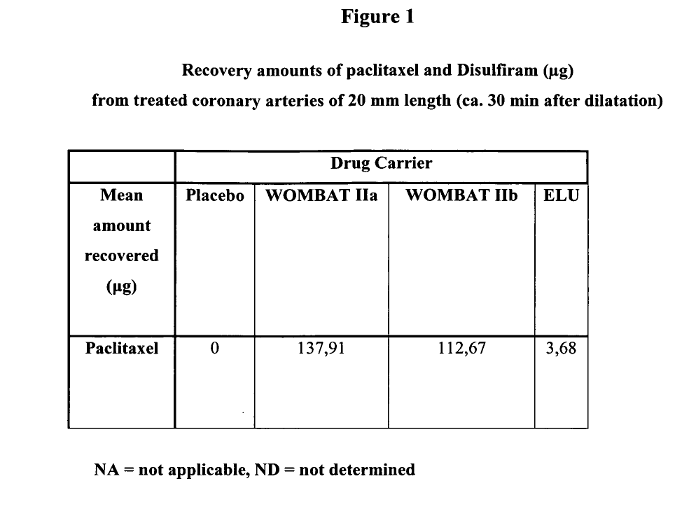

A hollow tube made from polyisoprene was coated as described in the examples below with a mixture of paclitaxel and a matrix component. In vivo data using coronary arteries from swine show the amount of paclitaxel substance that is effectively transferred into the artery tissue.

### EXAMPLES

### Example 1

### Production of Drug-Eluting Therapeutic Devices

The following therapeutic stock solutions on ethanol basis were prepared in order to be combined later to provide the final spray solution:

Paclitaxel: 40 mg/ml ethanol

Disulfiram: 40 mg/ml ethanol (soluble above approx. 35° C.)

### Production of the Spray-Solution:

For the production of the individual spray solutions, the stock solutions were combined as shown in table 1 below, diluted with absolute ethanol and combined with glycerol to give a final glycerol concentration of 5% (v/v).

For example:

Production of 20 ml of a solution comprising Disulfiram+Paclitaxel+glycerol which can be used for the spraying process and with which a coat can be sprayed onto e.g. expansible hollow tubes or balloon catheters to result in a surface concentration of 1 μg/mm² Disulfiram and 4 μg/mm² paclitaxel:

10 ml paclitaxel stock-solution (40 mg/ml)
+ 2.5 ml Disulfiram stock-solution
+ 1 ml glycerol
+ 6.5 ml ethanol abs.
mix well

An amount of 5% glycerol in combination with the addition of Disulfiram was shown to strongly improve the amount of paclitaxel that can be transferred from the surface of the therapeutic device (hollow part or balloon cathether) into the target tissue. Using paclitaxel at a concentration of 20 mg/ml and a total concentration of glycerol of about 5% (v/v), the molar ratio between glycerol and paclitaxel is about 30:1 which is considered to, without being bound by theory, permit a molecular segregation of individual paclitaxel molecules, thereby reducing the hydrophobic interaction of neighboring paclitaxel molecules resulting in an improved transfer rate and an increased amount transferred into tissue.

For the purpose of the invention any medical device can be coated with a therapeutic composition of the invention. For the following examples balloon catheters and expansible hollow tubes made of polyisoprene have been coated with compositions of the invention and the respective amount of therapeutic substance transferred into the target tissue has been examined.

Expansible drug-eluting hollow parts that can be used on a balloon catheter to deliver increased amounts of paclitaxel into tissue that is to be treated were spray-coated as follows:
(1) A flexible hollow tube made of polyisoprene as mentioned was slipped on the balloon part of a balloon catheter and affixed to the catheter.
(2) Upon complete expansion of the balloon using a standard inflation device, the inflation port of the balloon catheter was temporarily sealed and the inflation device was removed.
(3) The coating was applied to the hollow part in the expanded state using an ultrasonic sound spray system (Sono-Tek Corporation) and a therapeutic composition of the invention. In this method the therapeutic composition is dispersed using ultrasonic sound producing a fine spray of liquid which was applied onto the expanded hollow part under constant rotation of said part at 240 rpm.
(4) Step (3) was repeated a total of 3 times with intermittent drying phases to permit evaporation of the ethanol solvent from the surface.

Using spray solutions (see above) with a concentration of paclitaxel of 20 mg/ml a coating was obtained on the hollow parts with a paclitaxel concentration of 4±0.2 μg/mm² surface.

The therapeutic potential of the following coatings have been analyzed in vivo using a swine model system as outlined below:

TABLE 1

| Therapeutic device (hollow | Paclitaxel (surface | Matrix compound |
|---|---|---|
| part or balloon catheter) | concentration) | (surface concentration) |
| Wombat IIa | 4 μg/mm² | Disulfiram 1 μg/mm² |
| Wombat IIb | 4 μg/mm² | Disulfiram 4 μg/mm² |
| ELU | 2 μg/mm² | |

“ELU” refers to a commercial prior art balloon catheter coated with 2 μg/mm² paclitaxel.

1. A pharmaceutical composition comprising a taxane and an inhibitory compound capable of inhibiting the enzyme acetaldehyde dehydrogenase.

2. The pharmaceutical composition according to claim 1, wherein the inhibitory compound has the structure according to formula (I): 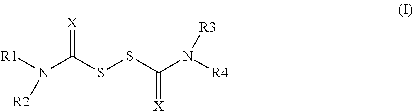
wherein
X is O or S; preferably S; and
R¹-R⁴ are each individually selected from the group consisting of C₁-C₆-alkyl, C₂-C₆-alkenyl, C₂-C₈-alkynyl, —(CH₂)ₙC(O)OH, —(CH₂)ₙC(O)OR⁵, —(CH₂)ₙOH, —(CH₂)ₙOR⁵, —(CH₂)ₙ-cycloalkyl, —(CH₂)ₙC(O)NH₂, —(CH₂)ₙC(O)NHR⁵, —(CH₂)ₙ-aryl, —(CH₂)ₙ-heteroaryl and —(CH₂)ₙNHR⁵; optionally substituted;
R⁵ is selected from the group consisting of hydrogen, C₁-C₆-alkyl, C₂-C₆-alkenyl, C₂-C₆-alkynyl, —(CH₂)ₙ-cycloalkyl and —(CH₂)ₙ-aryl; optionally substituted;
and
n is in each instance selected from 0, 1 and 2.

3. The pharmaceutical composition according to claim 1, wherein the inhibitory compound is coprin or disulfiram.

4. The pharmaceutical composition according to claim 1, wherein the molar ratio between the taxane and said inhibitory compound is between 3.5:1 and 0.07:1 and preferably between 1.4:1 and 0.3:1.

5. The pharmaceutical composition according to claim 1, wherein the taxane is paclitaxel or docetaxel.

6. The pharmaceutical composition according to claim 1, wherein the composition further comprises a polyol.

7. The pharmaceutical composition according to claim 6, wherein the polyol is an alcohol containing between 2 and 10 hydroxyl groups.

8. The pharmaceutical composition according to claim 7, wherein the polyol is selected from the group of alcohols consisting of pentaerythritol, glycerol, ethylene glycol and erythritol.

9. The pharmaceutical composition according to claim 1, wherein the molar ratio between the taxane and said polyol is between 1:3 to 1:250 and preferably between 1:6 to 1:120 and most preferably between 1:25 to 1:35.

10. A medical device comprising the pharmaceutical composition according to claim 1.

11. The medical device according to claim 10, wherein the device is an expansible hollow part, having at least one opening, which consists of an elastic biocompatible material that comprises elongated micro-cavities in its surface.

12. The medical device of claim 11, wherein more than 50% of the pharmaceutical composition is located in said micro-cavities.

13. The medical device of claim 10, wherein the elastic biocompatible material consists, comprises or essentially consists of a material selected from the group consisting of:
natural rubber, polyisoprene, a copolymer of isobutylene and isoprene, a halogenated butyl rubber , a polybutadiene, a styrene-butadiene rubber, a copolymer of polybutadiene and acrylonitrile, a hydrogenated nitrile rubber, a chloroprene rubber, a polychloroprene, latex, a neoprene, a baypren, latex, parylene, polyvalerolactone, poly-ε-decalactone, polylactic acid, polyglycol acid, polylactide, polyglycolide, co-polymer of polylactide and polyglycolide, poly-ε-caprolactone, polyhydroxy butyric acid, polyhydroxybutyrate, polyhydroxyvalerate, polyhydroxybutyrate-co-valerate, poly(1,4-dioxan-2,3-dione), poly(1,3-dioxan-2-one), poly-para-dioxanone, polyanhydride, polymaleicacidanhydride, polyhydroxymethacrylate, fibrin, polycyanoacrylate, polycaprolactondimethylacrylate, poly-β-maleic acid, polycaprolactonbutylacrylate, multiblockpolymers made of oligocaprolactondiole and oligodioxanondiole, polyetherestermultiblockpolymers made from PEG and polybutylenterephtalate, polypivotolactone, poly-glycolic acid trimethylcarbonate polycaprolactonglycolide, poly(g-ethyl glutamate), poly(dth-iminocarbonate), poly(dte-co-dt-carbonat), poly(bisphenol A-iminocarbonate), polyorthoester, poly-glycolic acid-trimethylcarbonate, polytrimethylcarbonate polyiminocarbonate, poly(n-vinyl)-pyrrolidone, polyvinylalcohols, polyesteramide, glycolized polyester, polyphosphoester, polyphosphazene, poly(p-carboxyphenoxy)propane], polyhydroxypentanoic acid, polyethylenoxidpropylenoxid, polyurethane, polyurethane comprising amino acids, polyetherester like polyethyleneoxide, polyalkeneoxalate, polyorthoester, lipids, carrageenane, fibrinogen, starch, collagene, protein-based polymers, polyaminoacids, zein, polyhydroxyalkanoate, pectic acid, actinic acid, carboxymethylsulfate, albumine, hyaluronic acid, chitosane, heparanesulfate, heparine, chondroitinsulfate, dextrane, β-cyclodextrine, copolymers comprising PEG and polypropyleneglycole, gummi arabicum, guar, gelatine, collagen-n-hydroxysuccinimide, phospholipids, polyacrylic acid, polyacrylate, polymethylmethacrylate, polybutylmethacrylate, polyacrylamide, polyacrylonitrile, polyamide, polyetheramide, polyethyleneamine, polyimide, polycarbonate, polycarbourethane, polyvinylketone, polyvinylhalogenide, polyvinylidenhalogenide, polyvinylether, polyisobutylene, aromatic compounds comprising a polyvinyl functional group, polyvinylester, polyvinylpyrollidone, polyoxymethylene, polytetramethyleneoxide, polyethylen, polypropylen, polytetrafluorethylen, polyetherurethane, silicon-polyetherurethane, silicon-polyurethane, silicon-polycarbonat-urethane, polyolefin-elastomers, epdm-rubber, fluorosilicone, carboxymethylchitosane, polyaryletheretherketone, polyetheretherketone, polyethylenterephtalate, polyvalerate, carboxymethylcellulose, cellulose, rayon, rayontriacetate, cellulosenitrate, celluloseacetate, hydroxyethylcellulose, cellulosebutyrate, celluloseacetatebutyrate, ethylvinylacetate, polysulfone, epoxy-resin, abs-resin, silicone like polysiloxane, polydimethylsiloxane, polyvinylhalogens, cellulose-ether, cellulose-triacetate, copolymers mixtures and derivatives thereof.

14. The medical device of claim 10, wherein at least part of said device is coated with said pharmaceutical composition such that the average taxane concentration on said coated surface is between 2 and 10 μg taxane/mm².

15. Pharmaceutical composition according to claim 1 for use in the treatment or prevention of diseases associated with or caused by hyperproliferation of cells.

16. Pharmaceutical composition of claim 15, wherein the disease is selected from stenosis, restenosis, stricture, defective bypass craft, thrombosis, dissection, tumor, calcification, arteriosclerosis, inflammation, autoimmune response, necrosis, injured anastomosis, lesion, allergy, wart, hyperproliferation, infection, scald, edema, coagulation, cicatrization, burn, frostbite and lymphangitis.
